(19) United States
(12) Patent Application Publication
(10) Pub. No.: US 2026/0151085 A1
(43) Pub. Date: Jun. 4, 2026

(54) WEARABLE ELECTRONIC DEVICE FOR MEASURING BIOMETRIC INFORMATION AND OPERATING METHOD THEREOF

(71) Applicant: Samsung Electronics Co., Ltd., Suwon-si (KR)

(72) Inventors: Hyunsu KIM, Suwon-si (KR); Seungwon LEE, Suwon-si (KR); Jaeyeong CHOI, Suwon-si (KR)

(21) Appl. No.: 19/454,724

(22) Filed: Jan. 21, 2026

Related U.S. Application Data
(63) Continuation of application No. PCT/KR2024/009685, filed on Jul. 8, 2024.

(30) Foreign Application Priority Data
Jul. 24, 2023 (KR) 10-2023-0096396
Oct. 18, 2023 (KR) 10-2023-0139378

(51) Int. Cl. A61B 5/00; A61B 5/0205; A61B 5/024; A61B 5/11

(52) U.S. Cl. CPC A61B 5/681; A61B 5/0205; A61B 5/11; A61B 5/6844; A61B 5/746; A61B 5/02438

(57) ABSTRACT

A wearable electronic device is provided. The wearable electronic device includes a first sensor, a second sensor, memory, comprising one or more storage media, storing instructions, and one or more processors communicatively coupled to the first sensor, the second sensor, and the memory, wherein the instructions, when executed by the one or more processors individually or collectively, cause the wearable electronic device to, a first state of the wearable electronic device set to measure a heart rate of a user at specified intervals, measure, using the first sensor, a first heart rate of the user during a first time, based on identifying that the first heart rate is outside a specified range, measure, using the first sensor, second heart rates of the user during a second time after the first time, and based on identifying that a ratio of heart rates outside the specified range among the second heart rates measured during the second time exceeds a specified ratio, output a notification indicating an abnormality in the heart rate of the user.

### CROSS-REFERENCE TO RELATED APPLICATION(S)

This application is a continuation application, claiming priority under 35 U.S.C. § 365(c), of an International application No. PCT/KR 2024/009685, filed on Jul. 8, 2024, which is based on and claims the benefit of a Korean patent application number 10-2023-0096396, filed on Jul. 24, 2023, in the Korean Intellectual Property Office, and of a Korean patent application number 10-2023-0139378, filed on Oct. 18, 2023, in the Korean Intellectual Property Office, the disclosure of each of which is incorporated by reference herein in its entirety.

### BACKGROUND

### 1. Field

The disclosure relates to a wearable electronic device for measuring bio-information and a method for operating the same.

### 2. Description of Related Art

Recent advancements in technology have led to the development of techniques for acquiring biosignals. In particular, technology is being developed to acquire a biosignal of a user through a wearable electronic device that includes a sensor capable of acquiring a biosignal, and to measure bio-information based on this.

For example, the wearable electronic device may include a photoplethysmography (PPG) sensor. The PPG sensor may include a light-emitting element and a light-receiving element. The PPG sensor may output light to the user's body using the light-emitting element and detect the light reflected by the user's body through the light-receiving element. The PPG sensor may acquire a PPG signal based on the light detected through the light-receiving element.

The wearable electronic device may measure bio-information such as the heart rate, pulse, saturation of peripheral oxygen (SpO2), or blood pressure of the user by analyzing the PPG signal acquired using the PPG sensor. Further, the wearable electronic device may provide measured bio-information of the user.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a wearable electronic device for measuring bio-information and a method for operating the same.

Additional aspects will be set forth in part in the description which follows and, in part, will be apparent from the description, or may be learned by practice of the presented embodiments.

In accordance with an aspect of the disclosure, a wearable electronic device is provided. The wearable electronic device includes a first sensor, a second sensor, memory, comprising one or more storage media, storing instructions, and one or more processors communicatively coupled to the first sensor, the second sensor, and the memory, wherein the instructions, when executed by the one or more processors individually or collectively, cause the wearable electronic device to, in a first state of the wearable electronic device set to measure a heart rate of a user at specified time intervals, measure, using the first sensor, a first heart rate of the user during a first time, based on identifying that the first heart rate is outside a specified range, measure, using the first sensor, second heart rates of the user during a second time after the first time, and based on identifying that a ratio of heart rates outside the specified range among the second heart rates measured during the second time exceeds a specified ratio, output a notification indicating an abnormality in the heart rate of the user.

In accordance with another aspect of the disclosure, a method performed by a wearable electronic device is provided. The method includes, in a first state of the wearable electronic device set to measure a heart rate of a user at specified time intervals, measuring, by the wearable electronic device using a first sensor included in the wearable electronic device, a first heart rate of the user during a first time, based on identifying that the first heart rate is outside a specified range, measuring, by the wearable electronic device using the first sensor, second heart rates of the user during a second time after the first time, and based on identifying that a ratio of heart rates outside the specified range among the second heart rates measured during the second time exceeds a specified ratio, outputting, by the wearable electronic device, a notification indicating an abnormality in the heart rate of the user.

In accordance with another aspect of the disclosure, one or more non-transitory computer-readable storage media storing one or more computer programs including computer-executable instructions that, when executed by one or more processors of a wearable electronic device individually or collectively, cause the wearable electronic device to perform operations are provided. The operations include, in a first state of the wearable electronic device set to measure a heart rate of a user at specified time intervals, measuring, by the wearable electronic device using a first sensor included in the wearable electronic device, a first heart rate of the user during a first time, based on identifying that the first heart rate is outside a specified range, measuring, by the wearable electronic device using the first sensor, second heart rates of the user during a second time after the first time, and based on identifying that a ratio of heart rates outside the specified range among the second heart rates measured during the second time exceeds a specified ratio, outputting, by the wearable electronic device, a notification indicating an abnormality in the heart rate of the user.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an electronic device in a network environment according to an embodiment of the disclosure;

FIG. 2A is a diagram illustrating a wearable electronic device worn by a user at rest according to an embodiment of the disclosure;

FIG. 2B is a schematic block diagram illustrating a configuration of a wearable electronic device according to an embodiment of the disclosure;

FIG. 3 is a schematic diagram illustrating a structure of a first sensor according to an embodiment of the disclosure;

FIG. 4 is a flowchart illustrating an operation of a wearable electronic device according to an embodiment of the disclosure;

FIG. 5 is a flowchart illustrating a method for starting measurement of a second heart rate by a wearable electronic device according to an embodiment of the disclosure;

FIG. 6 is a flowchart illustrating a method for outputting a notification based on a second heart rate by a wearable electronic device according to an embodiment of the disclosure;

FIG. 7 is a flowchart illustrating an operation of outputting a notification by a wearable electronic device according to an embodiment of the disclosure;

FIG. 8 is a flowchart illustrating an operation of, upon identifying a command for executing another function, stopping measurement of a second heart rate by a wearable electronic device according to an embodiment of the disclosure;

FIG. 9 is a diagram illustrating a notification provided by a wearable electronic device according to an embodiment of the disclosure;

FIG. 10 is a diagram illustrating information about a heart rate of a user provided by a wearable electronic device according to an embodiment of the disclosure;

FIG. 11 is a diagram illustrating a notification provided by an external electronic device communicatively connected to a wearable electronic device according to an embodiment of the disclosure; and

FIG. 12 is a diagram illustrating information about a heart rate of a user provided by an external electronic device communicatively connected to a wearable electronic device according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, description of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi™) chip, a Bluetooth™ chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

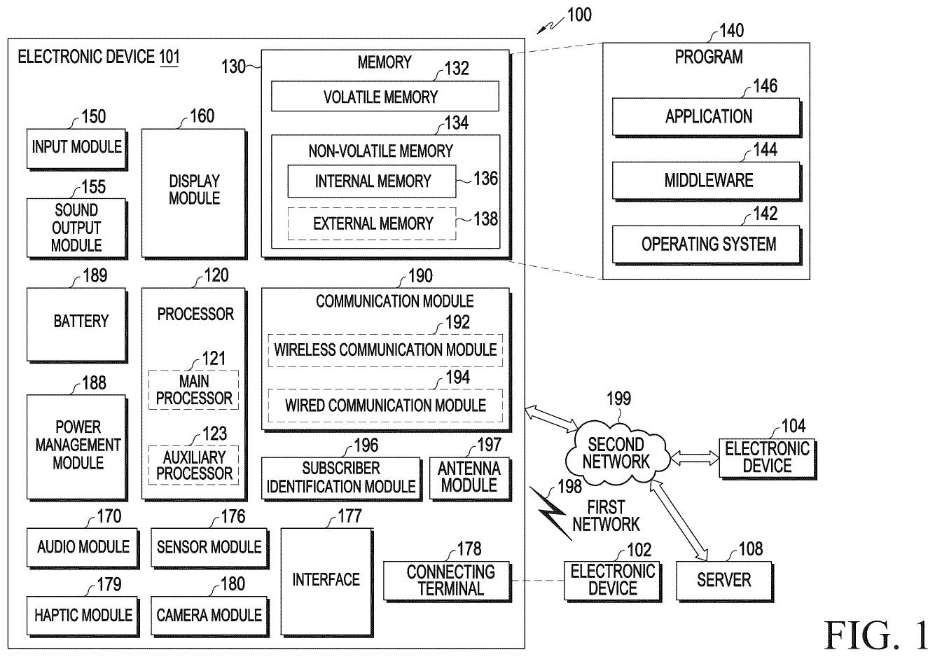

FIG. 1 is a block diagram illustrating an electronic device in a network environment according to an embodiment of the disclosure.

Referring to FIG. 1, an electronic device 101 in a network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134. The non-volatile memory 134 may include internal memory 136 and external memory 138.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the strength of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., the electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

The connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi™) direct, or infrared data association (IrDA)) or the second network 199 (e.g., a long-range communication network, such as a legacy cellular network, a fifth-generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth-generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to an embodiment, the antenna module 197 may form an mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices (e.g., the electronic devices 102 and 104 and the server 108). For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

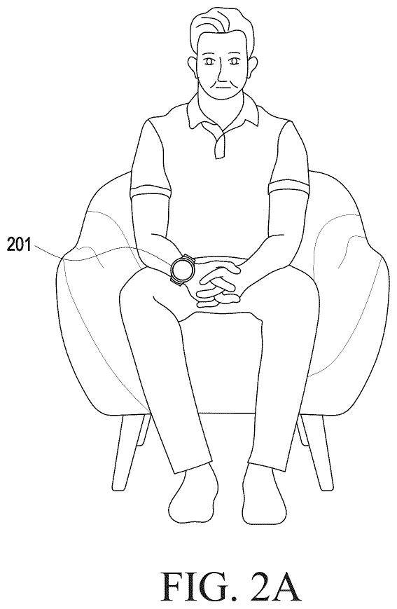

FIG. 2A is a diagram illustrating a wearable electronic device worn by a user at rest according to an embodiment of the disclosure.

Referring to FIG. 2A, according to an embodiment, a wearable electronic device 201 may be implemented as various types of electronic devices wearable by a user, such as a smart watch, a smart band, a smart ring, a wireless earphone, or smart glasses. According to an embodiment, the wearable electronic device 201 may be a device that may be worn on the user's wrist or a device that may be worn on another part (e.g., forearm, head, thigh, or the like) of the human body. According to an embodiment, the wearable electronic device 201 may be implemented as various types of electronic devices (e.g., a smartphone) that may perform communication functions, but are not in a form wearable by the user. According to an embodiment, the wearable electronic device 201 may be implemented the same as or similar to the electronic device 101 of FIG. 1.

According to an embodiment, the wearable electronic device 201 may measure bio-information of the user. For example, the wearable electronic device 201 may acquire a biosignal (e.g., a PPG signal) for measuring bio-information of a user 203 by outputting light from a sensor (e.g., a PPG sensor) included in the wearable electronic device 201. For example, the biosignal may refer to a signal corresponding to light that is output from a light-emitting portion of the sensor included in the wearable electronic device 201, reflected by the skin of the user 203, and at least partially received or acquired by a light-receiving portion of the sensor. For example, the biosignal may include a PPG signal or a PPG measurement signal. The wearable electronic device 201 may analyze the PPG signal to measure various pieces of bio-information (e.g., heart rate, pulse, saturation of peripheral oxygen (SpO2), or blood pressure) of the user.

According to an embodiment, the wearable electronic device 201 may identify whether the user is in a resting state. For example, when identifying that the user is in the resting state, the user's movement may be small or almost non-existent. According to an embodiment, the wearable electronic device 201 may identify the user's movement using a sensor (e.g., a motion sensor or an acceleration sensor) included in the wearable electronic device 201 and identify whether the user is at rest based on the identified movement. For example, when identifying that the user is at rest, the wearable electronic device 201 may not continuously measure the bio-information of the user. For example, the wearable electronic device 201 may measure the bio-information of the user at specified time intervals. Through this, the wearable electronic device 201 may reduce power consumption caused by the measurement of bio-information.

However, when bio-information of a user is set to be measured at specified time intervals, a conventional wearable electronic device may not be able to measure the bio-information of the user at times other than corresponding times. Accordingly, when a heart rate abnormality of the user occurs, the conventional wearable electronic device may not be able to provide an immediate notification to the user.

Upon detection of a heart rate abnormality in a state or mode of the wearable electronic device that is set to measure the heart rate of the user at specified time intervals, the wearable electronic device 201 according to an embodiment of the disclosure may continuously measure the user's heart rate during a specific time. Through this, the wearable electronic device 201 according to an embodiment may effectively provide a notification indicating the heart rate abnormality to the user.

FIG. 2B is a schematic block diagram illustrating the configuration of a wearable electronic device according to an embodiment of the disclosure.

Referring to FIG. 2B, according to an embodiment, a wearable electronic device 201 (e.g., the electronic device 101 of FIG. 1 or the wearable electronic device 201 of FIG. 2A) may include a processor 220, memory 230, a display 260, a first sensor 270, a second sensor 280, and a communication circuit 290.

According to an embodiment, the processor 220 may control the overall operation of the wearable electronic device 201. For example, the processor 220 may be implemented the same as or similar to the processor 120 of FIG. 1.

According to an embodiment, the processor 220 (e.g., the processor 120 of FIG. 1) may acquire a biosignal for measuring bio-information of the user through the first sensor 270.

According to an embodiment, the first sensor 270 may include a PPG sensor. According to an embodiment, the processor 220 may output first light to the user's body part (e.g., skin) using the first sensor 270. According to an embodiment, the processor 220 may acquire a signal (hereinafter, a biosignal) corresponding to the light reflected by the user's body part using the first sensor 270. For example, the processor 220 may acquire a PPG signal using the first sensor 270. According to an embodiment, the processor 220 may acquire or measure bio-information including the heart rate, oxygen saturation, stress, arrhythmia, and/or blood pressure of the user, based on the acquired biosignal (e.g., PPG signal).

Depending on implementation, the first sensor 270 may include a plurality of electrodes. The plurality of electrodes may be used to directly contact the user's skin to sense or detect a voltage corresponding to electrical resistance or a voltage corresponding to electrical conductivity. According to an embodiment, the processor 220 may acquire electrodermal activity (EDA) including galvanic skin reflex (GSR), a bioelectrical impedance analysis (BIA) signal, and/or an electrocardiogram (ECG) signal using the first sensor 270. According to an embodiment, the processor 220 may measure ECG including an ECG and/or body fat based on the biosignal. However, the first sensor 270 is not limited to this and may include various types of sensors capable of acquiring or measuring bio-information of the user.

According to an embodiment, the processor 220 may acquire biosignals at predetermined time intervals or continuously during a specific time using the first sensor 270.

According to an embodiment, the processor 220 may measure a first heart rate of the user during a first time (e.g., 30 seconds) using the first sensor 270 in a first state of the wearable electronic device 201 (e.g., a state where the wearable electronic device 201 is operating in the resting mode) set to measure the heart rate of the user at specified time intervals (e.g., every 10 minutes). For example, the processor 220 may measure the heart rate of the user every 10 minutes, while operating in the resting mode. The first heart rate may indicate the heart rate of the user measured in the first state (e.g., operating in the resting mode).

According to an embodiment, the processor 220 may identify whether the first heart rate is outside a specified range. For example, the specified range may be set as a normal heart rate range. For example, the specified range may indicate a heart rate range lower than a first reference value indicating a high heart rate and higher than a second reference value (e.g., a value smaller than the first reference value) indicating a low heart rate. For example, the first reference value and the second reference value may be values preset by the user or by the processor 220. In addition, the first reference value and the second reference value may be determined based on personal information (e.g., height, weight, gender, and/or body fat) of the user.

According to an embodiment, based on identifying that the first heart rate is outside the specified range, the processor 220 may measure a second heart rate of the user using the first sensor 270 during a second time after the first time. At this time, the processor 220 may continuously measure the heart rate of the user during a specified second time (e.g., 10 minutes). For example, the second heart rate may indicate the heart rate of the user continuously measured during the second time. In addition, the second heart rate may include a plurality of heart rate values continuously measured during the second time.

According to an embodiment, the processor 220 may identify whether a ratio of heart rates outside the specified range to second heart rates measured during the second time exceeds a specified ratio. For example, the specified ratio may indicate a ratio (e.g., 80%) for determining a heart rate abnormality of the user. The specified ratio may be set by the user or automatically by the processor 220.

According to an embodiment, when identifying that a ratio of heart rates outside the specified range to the second heart rates measured during the second time exceeds the specified ratio, the processor 220 may output a notification indicating a heart rate abnormality of the user. For example, the processor 220 may display the notification indicating the heart rate abnormality through the display 260. The processor 220 may display information about the measured heart rates through the display 260. Depending on implementation, the processor 220 may output the notification indicating the heart rate abnormality through a speaker (not shown).

According to an embodiment, the processor 220 may store a history of notifications indicating the heart rate abnormality in the memory 230. In addition, the processor 220 may store information about the measured heart rates in the memory 230.

According to an embodiment, the processor 220 may transmit information about the measured heart rates to an external electronic device (e.g., a terminal communicatively connected to the wearable electronic device 201) through the communication circuit 290. In addition, the processor 220 may also transmit a control signal to the external electronic device through the communication circuit 290 to cause the external electronic device to output a notification indicating the heart rate abnormality.

According to an embodiment, the second sensor 280 may acquire a sensing value corresponding to movement of the user wearing the wearable electronic device 201. The second sensor 280 may be implemented the same as or similar to the sensor module 176 of FIG. 1. For example, the second sensor 280 may include at least one of an acceleration sensor, an inertia sensor, a motion sensor, an IR sensor, a temperature (body temperature) sensor, a gyro sensor, a gravity sensor or geomagnetic sensor, a proximity sensor, an illuminance sensor, a time of flight (TOF) sensor, or a barometer sensor. However, the second sensor 280 is not limited to this and may include various types of sensors capable of identifying the user's movement.

According to an embodiment, the processor 220 may identify whether the user is at rest using the second sensor 280. In addition, the processor 220 may also identify whether the user is exercising using the second sensor 280. For example, the processor 220 may compare a sensing value indicating the user's movement wearing the wearable electronic device 201 with specified values, and determine whether the user is at or exercising according to a comparison result. For example, when the sensing value indicating the user's movement is greater than a first specified value, the processor 200 may determine that the user is exercising. Alternatively, when the sensing value indicating the user's movement is smaller than a second specified value (e.g., a value smaller than the first specified value), the processor 220 may determine that the user is at rest.

According to an embodiment, the processor 220 may display the bio-information (e.g., heart rate) of the user measured using the first sensor 270 on the display 260 (e.g., the display module 160 of FIG. 1). In addition, the processor 220 may display the notification indicating the heart rate abnormality on the display 260 (e.g., the display module 160 of FIG. 1). For example, the notification may include at least one of a heart rate value corresponding to the heart rate abnormality, information that describes the heart rate abnormality, or information about emergency measures.

According to an embodiment, the processor 220 may transmit the bio-information of the user measured using the first sensor 270 to an external electronic device (not shown) through the communication circuit 290 (e.g., the communication module 190 of FIG. 1).

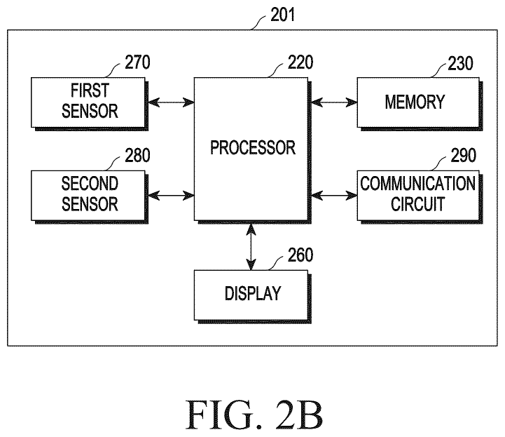

FIG. 3 is a schematic diagram illustrating the structure of a first sensor according to an embodiment of the disclosure.

Referring to FIG. 3, according to an embodiment, a first sensor (e.g., the first sensor 270 of FIG. 2B) may be disposed on the rear surface (e.g., a portion that contacts the user's wrist) of the wearable electronic device 201. For example, the first sensor 270 may be implemented as a PPG sensor.

According to an embodiment, the first sensor 270 may include a plurality of light-emitting elements 271 and 272 and a plurality of light-receiving elements 276, 277, and 278. For example, the plurality of light-emitting elements 271 and 272 may include a light-emitting diode (LED). For example, the plurality of light-receiving elements 276, 277, and 278 may include at least one of an avalanche photodiode (APD), a single photon avalanche diode (SPAD), a photodiode, a photomultiplier tube (PMT), a charge coupled device (CCD), a CMOS array, or a spectrometer. Although the numbers, shapes, sizes, types, and positions of the light-emitting elements and the light-receiving elements are specified and illustrated in FIG. 3 for convenience of description, the numbers, shapes, sizes, types, and positions of the light-emitting elements and the light-receiving elements are not limited to these and may be implemented in various manners.

According to an embodiment, each of the plurality of light-emitting elements 271 and 272 may output light to the body part of the user wearing the wearable electronic device 201 (e.g., the user's skin in contact with the wearable electronic device 201). For example, the light may include at least one of red light, green light, blue light, or IR light. For example, the wearable electronic device 201 may use light in the green wavelength band to measure a heart rate. Since light in the green wavelength band penetrates relatively shallowly into the user's skin, the wearable electronic device 201 may acquire a biosignal with less noise. According to an embodiment, the wearable electronic device 201 may use light in the red wavelength band to measure a heart rate. Since light in the red wavelength band penetrates relatively deeply into the user's skin, the wearable electronic device 201 may measure a more accurate heart rate. According to an embodiment, the wearable electronic device 201 may measure a heart rate and oxygen saturation (SpO2) using light in the IR wavelength band. The wearable electronic device 201 may measure relatively various pieces of bio-information using light in the IR wavelength band compared to using light in other wavelength bands. According to an embodiment, the wearable electronic device 201 may measure the user's skin tone using light in the IR wavelength band, red wavelength band, and green wavelength band. According to an embodiment, the wearable electronic device 201 may also measure the blood glucose of the user using light in the blue wavelength band. The wearable electronic device 201 may measure bio-information using light in an appropriate wavelength band.

According to an embodiment, the plurality of light-receiving elements 276, 277, and 278 may receive at least a portion of light that is output from the plurality of light-emitting elements 271 and 272 and reflected or transmitted by the user's body tissue (e.g., skin, skin tissue, fat layer, vein, artery, and/or capillary).

According to an embodiment, although not shown, the wearable electronic device 201 may include an analog-to-digital converter (ADC). According to an embodiment, the wearable electronic device 201 may convert a signal output from the plurality of light-receiving elements 276, 277, and 278 from an analog signal to a digital signal via the ADC. According to an embodiment, the ADC may be separately provided between the light-receiving portion 320 and the processor 220.

According to an embodiment, the processor 220 may acquire the digital signal converted by the ADC as a biosignal of the user. The processor 220 may analyze the biosignal to measure or acquire bio-information (e.g., heart rate) of the user.

According to an embodiment, the wearable electronic device 201 may further include a plurality of electrodes 251, 252, and 253. For example, a first electrode 251 and a second electrode 252 may be disposed on the rear surface (e.g., a portion that contacts the user's wrist) of the wearable electronic device 201. A third electrode 253 may be disposed on a side portion (e.g., on a button located on the side portion) of the wearable electronic device 201. The wearable electronic device 201 may acquire a biosignal (e.g., EDA including GSR, a BIA signal, and/or an ECG signal) for measuring bio-information (e.g., electrocardiogram, skin conductivity, or body composition) of the user using the plurality of electrodes 251, 252, and 253. The processor 220 may analyze the biosignal to measure or acquire the bio-information (e.g., electrocardiogram, skin conductivity, or body composition) of the user.

Operations of the wearable electronic device 201 described below may be performed or controlled by the processor 220. However, for convenience of description, the operations performed or controlled by the processor 220 will be described as being performed by the wearable electronic device 201.

For the operations of the wearable electronic device 201, an operation of measuring the heart rate of the user will be primarily focused on. However, the technical idea of the disclosure may be applied not only to the operation of measuring the heart rate of the user but also to an operation of measuring various pieces of bio-information of the user.

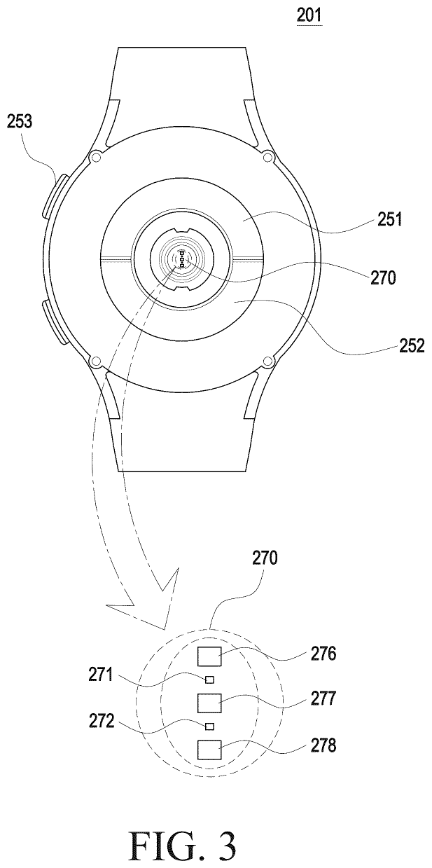

FIG. 4 is a flowchart illustrating an operation of a wearable electronic device according to an embodiment of the disclosure.

Referring to FIG. 4, according to an embodiment, in operation 401, a wearable electronic device (e.g., the wearable electronic device 201 of FIG. 2A) may measure a first heart rate of a user during a first time (e.g., 30 seconds) using a first sensor (e.g., the first sensor 270 of FIG. 2B) in a first state (e.g., a state where it operates in a resting mode) set to measure the heart rate of the user at specified time intervals. For example, when a specified time arrives, the wearable electronic device 201 may measure a first heart rate of the user during the first time, using the first sensor 270.

According to an embodiment, in operation 403, based on identifying that the first heart rate is outside a specified range, the wearable electronic device 201 may measure a second heart rate of the user during a second time (e.g., 10 minutes) after the first time, using the first sensor 270. For example, when identifying that the first heart rate is outside the specified range, the wearable electronic device 201 may continuously measure second heart rates of the user for a longer time. For example, the specified range may indicate a normal heart rate range.

According to an embodiment, in operation 405, when identifying that the ratio of heart rates outside the specified range to the second heart rates measured during the second time exceeds a specified ratio (e.g., 80%), the wearable electronic device 201 may output a notification indicating a heart rate abnormality of the user. For example, the specified ratio may be set as a ratio for determining a heart rate abnormality. For example, the wearable electronic device 201 may output the notification indicating the heart rate abnormality of the user through auditory, visual, or haptic (e.g., vibration) means.

According to an embodiment, in operation 407, the wearable electronic device 201 may store information about the measured heart rates and a notification history in memory (e.g., the memory 230 of FIG. 2B). In addition, the wearable electronic device 201 may transmit information about the measured heart rates and the notification history to an external electronic device connected to the wearable electronic device by communication (e.g., a connection based on Bluetooth^{TM} communication).

According to the method described above, upon detection of a heart rate abnormality in the state or mode of the wearable electronic device set to measure the heart rate of the user at specified time intervals, the wearable electronic device 201 may continuously measure the heart rate of the user during a specific time. Through this, the wearable electronic device 201 according to an embodiment may effectively provide a notification indicating the heart rate abnormality to the user.

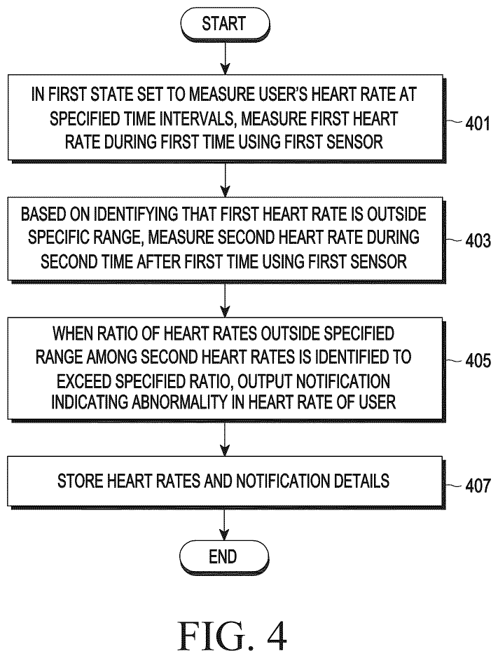

FIG. 5 is a flowchart illustrating a method for starting measurement of a second heart rate by a wearable electronic device according to an embodiment of the disclosure.

Referring to FIG. 5, according to an embodiment, a wearable electronic device (e.g., the wearable electronic device 201 of FIG. 2A) may identify whether a user is at rest in a first state set to measure the heart rate of the user at specified time intervals. According to an embodiment, in operation 501, the wearable electronic device (e.g., the wearable electronic device 201 of FIG. 2A) may identify the user's movement using a second sensor (e.g., the second sensor 280 of FIG. 2B).

According to an embodiment, in operation 503, the wearable electronic device 201 may identify whether the user is at rest, based on the identified user movement. For example, when identifying that a sensing value indicating the user movement sensed by the second sensor 280 is smaller than a specified value, the wearable electronic device 201 may identify that the user is at rest. Alternatively, when identifying that the wearable electronic device 201 is operating in the resting mode, the wearable electronic device 201 may identify that the user is at rest.

According to an embodiment, when identifying that the user is not at rest (no in operation 503), the wearable electronic device 201 may identify again whether the user is at rest.

According to an embodiment, when identifying that the user is at rest (yes in operation 503), the wearable electronic device 201 may identify whether a specified time (e.g., 0 minutes, 10 minutes, 20 minutes, 30 minutes, 40 minutes, and 50 minutes of every hour) set to measure the heart rate of the user has arrived, in operation 505. The wearable electronic device 201 may identify whether the specified time set to measure the heart rate of the user has arrived using a preset timer.

According to an embodiment, when identifying that the specified time has not arrived (no in operation 505), the wearable electronic device 201 may identify again whether the user is at rest.

According to an embodiment, when identifying that the specified time has arrived (yes in operation 505), the wearable electronic device 201 may acquire a first biosignal and measure a first heart rate during a specified first time (e.g., 30 seconds) using the first sensor 270, in operation 507. For example, the first biosignal may include a PPG signal acquired during the first time.

According to an embodiment, in operation 509, the wearable electronic device 201 may identify the signal to noise ratio (SNR) of the first biosignal. In addition, the wearable electronic device 201 may identify whether the SNR of the first biosignal is greater than a specified value. For example, when identifying that the SNR of the first biosignal is greater than the specified value, the wearable electronic device 201 may determine that the first biosignal is a signal of sufficient quality to measure the heart rate of the user. For example, the specified value may indicate a reference value for determining whether the first biosignal is a signal of sufficient quality to measure the heart rate of the user. For example, when the heart rate is measured as 60 bpm, the wearable electronic device 201 may determine an area corresponding to 60 bpm+5 bpm to 60 bpm−5 bpm as a signal area, and the remaining area as a noise area. For example, the value of the signal area/(signal area+noise area) of the first biosignal may be an SNR, and the specified value may be set to 0.2.

Depending on implementation, when the peak of the waveform of the first biosignal is determined to be the same as or similar to a specified type of peak, the wearable electronic device 201 may determine that the first biosignal is a signal of sufficient quality to measure the heart rate of the user.

According to an embodiment, when identifying that the SNR of the first biosignal is not greater than the specified value (no in operation 509), the wearable electronic device 201 may identify again whether the user is at rest.

According to an embodiment, when identifying that the SNR of the first biosignal is greater than the specified value (yes in operation 509), the wearable electronic device 201 may identify whether the measured first heart rate is outside a specified range, in operation 511. For example, the specified range may be a normal heart rate range.

According to an embodiment, when identifying that the first heart rate is not outside the specified range (no in operation 511), the wearable electronic device 201 may identify again whether the user is at rest.

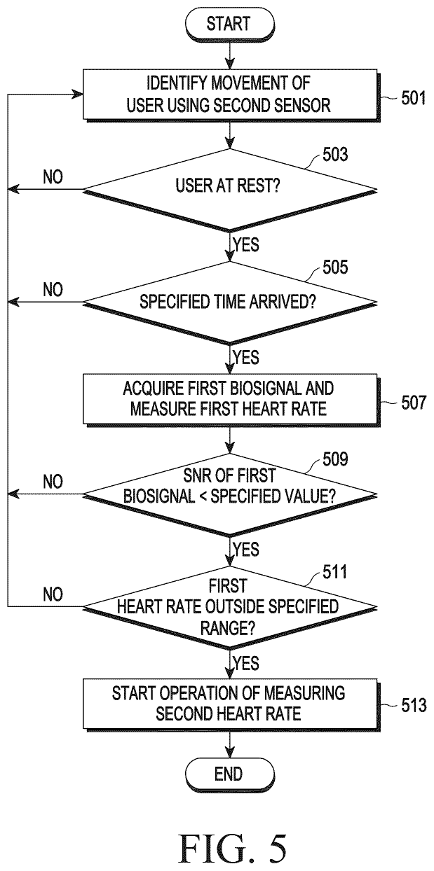

According to an embodiment, when identifying that the first heart rate is outside the specified range (yes in operation 511), the wearable electronic device 201 may start an operation of measuring a second heart rate, in operation 513. For example, when identifying that the first heart rate is outside the specified range, the wearable electronic device 201 may start an operation of continuously identifying or checking the heart rate of the user for a longer time. The operation of measuring a second heart rate by the wearable electronic device 201 will be described below in more detail with reference to FIG. 6.

FIG. 6 is a flowchart illustrating a method for outputting a notification based on a second heart rate by a wearable electronic device according to an embodiment of the disclosure.

Referring to FIG. 6, according to an embodiment, in operation 601, a wearable electronic device (e.g., the wearable electronic device 201 of FIG. 2A) may start an operation of continuously measuring second heart rates of a user during a second time (e.g., 10 minutes) using a first sensor (e.g., the first sensor 270 of FIG. 2B).

According to an embodiment, in operation 603, the wearable electronic device 201 may identify whether the measurement of the second heart rates is for identifying a low heart rate. For example, when identifying that a first heart rate is lower than a second reference value (e.g., 50 bpm) indicating a low heart rate, the wearable electronic device 201 may perform an operation of checking or re-identifying the low heart rate. Alternatively, when identifying that the first heart rate is higher than a first reference value (e.g., 120 bpm) indicating a high heart rate, the wearable electronic device 201 may perform an operation of checking or re-identifying the high heart rate.

According to an embodiment, when identifying that the measurement of the second heart rates is not for identifying a low heart rate (no in operation 603), the wearable electronic device 201 may identify the user's movement using a second sensor (e.g., the second sensor 280 of FIG. 2B), in operation 605. For example, when identifying that the measurement of the second heart rates is not for identifying a low heart rate, the wearable electronic device 201 may determine that the measurement of the second heart rates is for identifying a high heart rate. Since the heart rate of the user may temporarily increase when the user is moving a lot, the wearable electronic device 201 may identify the degree of the user's movement.

According to an embodiment, in operation 607, the wearable electronic device 201 may identify whether a sensing value indicating the user's movement is greater than a specified value. The specified value may include a value indicating that the user is in an exercising state. For example, the wearable electronic device 201 may identify whether the sensing value indicating the user's movement is greater than the specified value during a specific time (e.g., a time period from a time point a specific time before a current time point to a time point the specific time after the current point, or a time period spanning the specific time from the current time point).

According to an embodiment, when identifying that the sensing value indicating the user's movement is greater than the specified value (yes in operation 607), the wearable electronic device 201 may stop the operation of measuring second heart rates, in operation 617. In this case, the wearable electronic device 201 may determine that there is no need to continue checking the second heart rates. Further, the wearable electronic device 201 may not output a notification indicating a heart rate abnormality of the user.

According to an embodiment, when identifying that the sensing value indicating the user's movement is not greater than the specified value (no in operation 607), the wearable electronic device 201 may identify the wearing state of the wearable electronic device 201, in operation 609. According to an embodiment, when identifying that the measurement of the second heart rates is for identifying a low heart rate (yes in operation 603), the wearable electronic device 201 may identify the wearing state of the wearable electronic device 201, in operation 609. For example, the wearable electronic device 201 may identify whether the wearable electronic device 201 is being worn by the user using the first sensor 270.

According to an embodiment, in operation 611, the wearable electronic device 201 may identify whether the wearing state of the wearable electronic device 201 satisfies a specified condition. For example, the specified condition may be a condition indicating whether a wearing state sufficient to measure the heart rate of the user is maintained during the second time. For example, the wearable electronic device 201 may identify whether the wearable electronic device 201 remains worn by the user during the second time for measuring second heart rates.

According to an embodiment, when identifying that the wearing state of the wearable electronic device 201 does not satisfy the specified condition (no in operation 611), the wearable electronic device 201 may stop the operation of measuring second heart rates, in operation 617. In this case, the wearable electronic device 201 may determine that there is no need to continue checking the second heart rates. Further, the wearable electronic device 201 may not output a notification indicating a heart rate abnormality of the user.

According to an embodiment, when identifying that the wearing state of the wearable electronic device 201 satisfies the specified condition (yes in operation 611), the wearable electronic device 201 may identify whether the ratio of heart rates outside the specified range to the second heart rates measured during the second time exceeds a specified ratio, in operation 613.

According to an embodiment, when identifying that the ratio of heart rates outside the specified range to the second heart rates measured during the second time exceeds the specified ratio (yes in operation 613), the wearable electronic device 201 may output a notification indicating a heart rate abnormality of the user, in operation 615. After outputting the notification, the wearable electronic device 201 may continue to monitor the heart rate of the user. For example, after outputting the notification, the wearable electronic device 201 may sequentially perform the operations starting again from operation 601. That is, after outputting the notification, the wearable electronic device 201 may continuously measure the heart rate of the user during a specified time (e.g., the second time).

According to an embodiment, when identifying that the ratio of heart rates outside the specified range to the second heart rates measured during the second time does not exceed the specified ratio (no in operation 613), the wearable electronic device 201 may stop the operation of measuring second heart rates, in operation 617. In this case, the wearable electronic device 201 may determine that there is no need to continue checking the second heart rates. Further, the wearable electronic device 201 may not output a notification indicating a heart rate abnormality of the user. For example, after deciding not to output a notification, the wearable electronic device 201 may sequentially perform the operations again starting from operation 501 of FIG. 5. That is, after deciding not to output a notification, the wearable electronic device 201 may measure the heart rate of the user again at the specified time intervals.

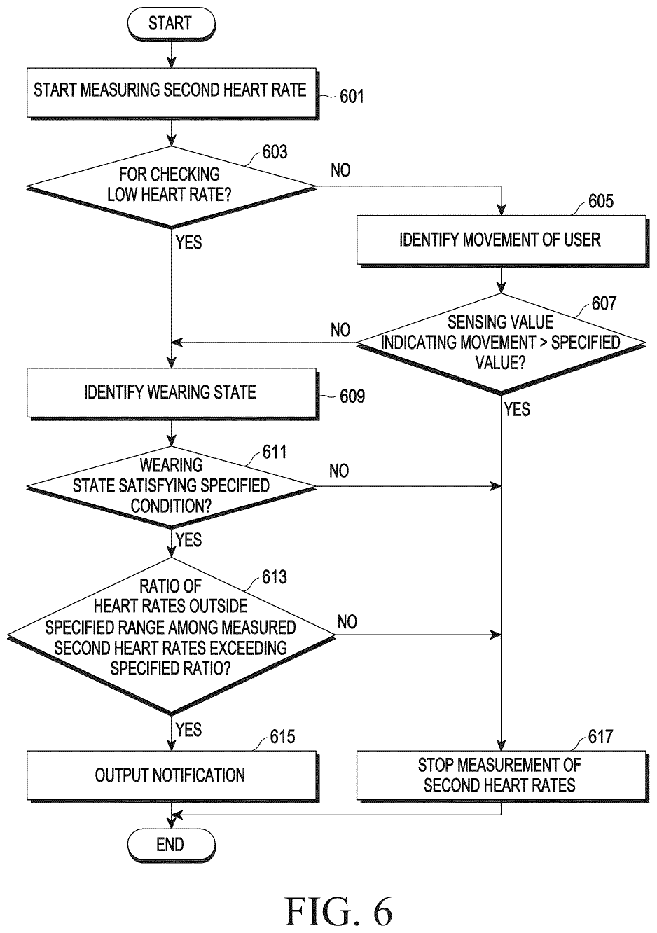

FIG. 7 is a flowchart illustrating an operation of outputting a notification by a wearable electronic device according to an embodiment of the disclosure.

Referring to FIG. 7, according to an embodiment, in operation 701, a wearable electronic device (e.g., the wearable electronic device 201 of FIG. 2A) may measure second heart rates of a user during a second time (e.g., 10 minutes) using a first sensor (e.g., the first sensor 270 of FIG. 2B).

According to an embodiment, in operation 703, the wearable electronic device 201 may identify whether the measurement of second heart rates is for identifying a low heart rate. For example, when identifying that a first heart rate is lower than a second reference value (e.g., 50 bpm) indicating a low heart rate, the wearable electronic device 201 may perform an operation of checking or re-identifying the low heart rate. Alternatively, when identifying that the first heart rate is higher than a first reference value (e.g., 120 bpm) indicating a high heart rate, the wearable electronic device 201 may perform an operation of checking or re-identifying the high heart rate.

According to an embodiment, when identifying that the measurement of second heart rates is not for identifying a low heart rate (i.e., for identifying a high heart rate) (no in operation 703), the wearable electronic device 201 may provide information indicating the high heart rate of the user through a notification, when the ratio of heart rates higher than the first reference value to the second heart rates exceeds a specified ratio, in operation 705.

According to an embodiment, when identifying that the measurement of second heart rates is for identifying a low heart rate (yes in operation 703), the wearable electronic device 201 may provide information indicating the low heart rate of the user through a notification, when the ratio of heart rates lower than the second reference value to the second heart rates exceeds a specified ratio, in operation 707.

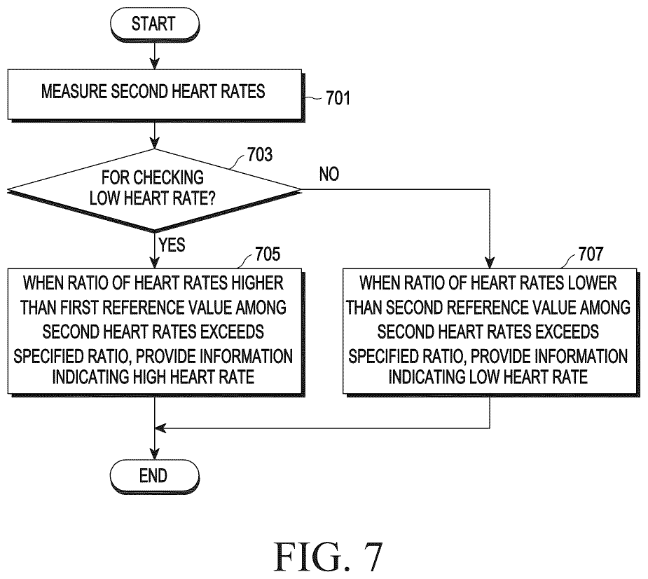

FIG. 8 is a flowchart illustrating an operation of, upon identifying a command to execute another function, stopping measurement of a second heart rate by a wearable electronic device according to an embodiment of the disclosure.

Referring to FIG. 8, according to an embodiment, in operation 801, a wearable electronic device (e.g., the wearable electronic device 201 of FIG. 2A) may measure second heart rates during the second time using the first sensor (e.g., the first sensor 270 of FIG. 2B).

According to an embodiment, in operation 803, while measuring the second heart rates, the wearable electronic device 201 may identify whether execution of another function of the wearable electronic device 201 has been commanded. For example, the other function may include a function related to an exercise mode of the wearable electronic device 201. Alternatively, the other function may include a function of measuring bio-information other than the heart rate using the first sensor 270 of the wearable electronic device 201.

According to an embodiment, when identifying that the execution of the other function has not been commanded (no in operation 803), the wearable electronic device 201 may continue to measure second heart rates during the second time, in operation 805. According to another embodiment, when identifying a command for executing a function of the wearable electronic device unrelated to measuring second heart rates, the wearable electronic device 201 may continue to measure the second heart rates during the second time.

According to an embodiment, when identifying that the execution of another function has been commanded (yes in operation 803), the wearable electronic device 201 may stop the measurement of second heart rates, in operation 807. For example, the wearable electronic device 201 may output a notification indicating the cessation of the second heart rate measurement (e.g., through visual means, auditory means, and/or haptic means). For example, when the user starts exercising, the heart rate of the user increases due to the exercise, and thus, the wearable electronic device 201 may not accurately measure the heart rate of the user. In addition, when measuring other bio-information of the user using the first sensor 270, the wearable electronic device 201 may not accurately measure the heart rate of the user. Accordingly, when another function of the wearable electronic device 201 is executed, the wearable electronic device 201 may determine that there is no need to continue checking the second heart rates.

According to another embodiment, when identifying that another function of the wearable electronic device 201 is already running in the step of starting the measurement of second heart rates, the wearable electronic device 201 may not start the measurement of second heart rates.

According to another embodiment, when a function of the wearable electronic device unrelated to measuring second heart rates is running, the wearable electronic device 201 may continue to measure the second heart rates during the second time.

According to the method described above, when a heart rate abnormality is detected in a state or mode of the wearable electronic device set to measure the heart rate of the user at specified time intervals, the wearable electronic device 201 may continuously measure the heart rate of the user during a specific time. Through this, the wearable electronic device 201 according to an embodiment may effectively provide a notification indicating the heart rate abnormality to the user.

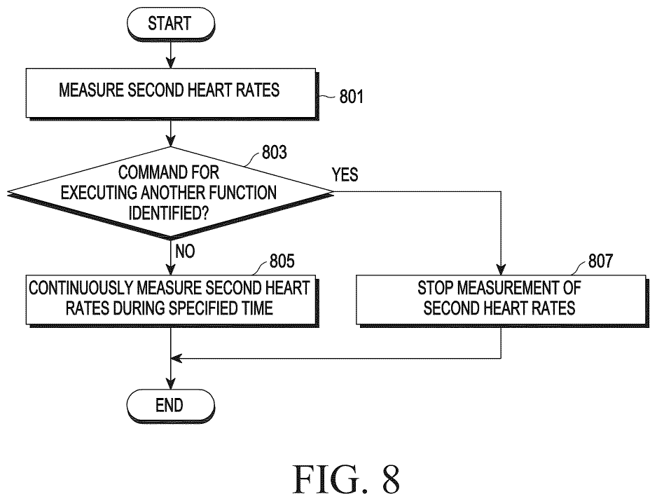

FIG. 9 is a diagram illustrating a notification provided by a wearable electronic device according to an embodiment of the disclosure.

Referring to FIG. 9, according to an embodiment, a wearable electronic device (e.g., the wearable electronic device 201 of FIG. 2A) may display a notification 910 indicating a heart rate abnormality of a user on a display (e.g., the display 260 of FIG. 2B), based on a result of measuring second heart rates. For example, the notification 910 may include information about a heart rate value (e.g., 125bmp) corresponding to the heart rate abnormality and/or information about heart rate values measured during a second time. For example, the wearable electronic device 201 may display the heart rate values measured during the second time as graphical information using a graph and/or dots. Depending on implementation, the notification 910 may further include a guide (e.g., health-related information) helpful for the heart rate abnormality.

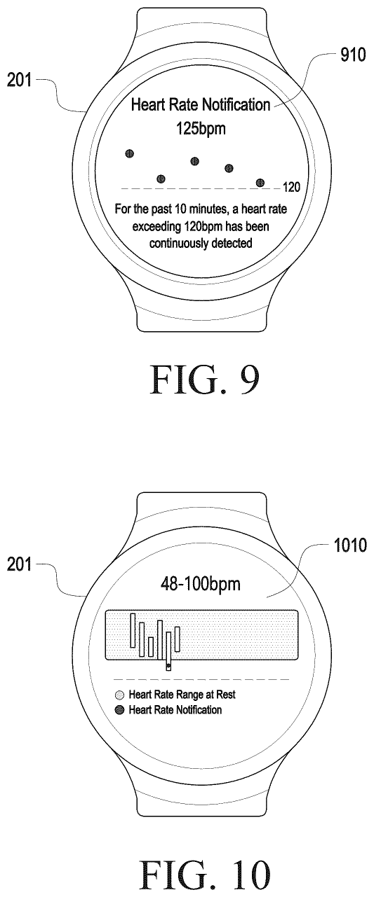

FIG. 10 is a diagram illustrating information about a heart rate of a user provided by a wearable electronic device according to an embodiment of the disclosure.

Referring to FIG. 10, according to an embodiment, a wearable electronic device (e.g., the wearable electronic device 201 of FIG. 2A) may store information indicating a result of measuring second heart rates. In addition, the wearable electronic device 201 may store notification output details.

According to an embodiment, the wearable electronic device may display information 1010 indicating the result of measuring second heart rates on a display (e.g., the display 260 of FIG. 2B). For example, the information 1010 indicating the result of measuring second heart rates may include information about heart rate values measured during the second time (e.g., numerical values for a measured heart rate range or heart rate values graphically represented using a graph). For example, the information 1010 indicating the result of measuring second heart rates may indicate the trend of heart rate changes over time. In addition, the information 1010 indicating the result of measuring second heart rates may display a history of notifications indicating heart rate abnormalities through a separate object (e.g., dots).

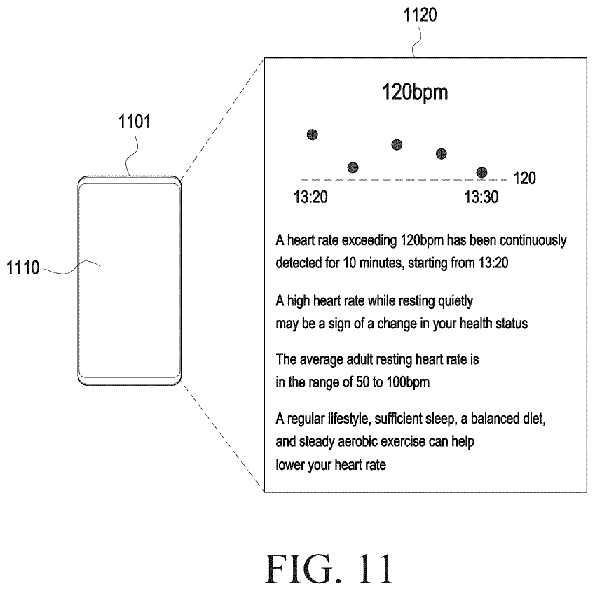

FIG. 11 is a diagram illustrating a notification provided by an external electronic device communicatively connected to a wearable electronic device according to an embodiment of the disclosure.

Referring to FIG. 11, according to an embodiment, a wearable electronic device (e.g., the wearable electronic device 201 of FIG. 2A) may transmit information about a notification indicating a heart rate abnormality of a user to an external electronic device 1101.

According to an embodiment, the external electronic device 1101 may display a notification 1120 indicating the heart rate abnormality on a display 1110 of the external electronic device 1101. For example, the notification 1120 may include information about a heart rate value (e.g., 120 bpm) corresponding to the heart rate abnormality and/or information about heart rate values measured during a second time. For example, the wearable electronic device 201 may display the heart rate values measured during the second time as graphical information using a graph and/or dots. In addition, the notification 1120 may further include a guide (e.g., health-related information) helpful for the heart rate abnormality.

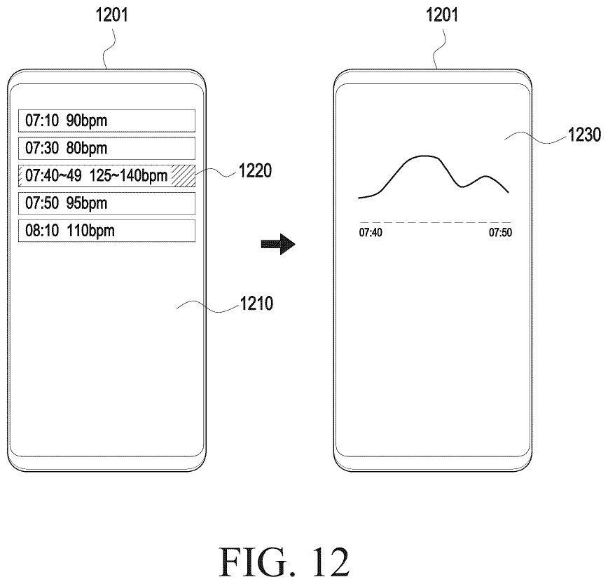

FIG. 12 is a diagram illustrating information about a heart rate of a user provided by an external electronic device communicatively connected to a wearable electronic device according to an embodiment of the disclosure.

Referring to FIG. 12, according to an embodiment, a wearable electronic device (e.g., the wearable electronic device 201 of FIG. 2A) may transmit information about a result of measuring the heart rate of the user to an external electronic device 1201.

According to an embodiment, the external electronic device 1201 may display information 1210 about measured heart rate values on a time period basis. For example, it may also display information 1220 about heart rate values (e.g., second heart rate values) continuously measured during a specified time period from a specific time.

According to an embodiment, when identifying a user input for a specific time, the external electronic device 1201 may display detailed information 1230 about heart rate values continuously measured during a specific time period. For example, the detailed information 1230 may include a graph illustrating the trend of the heart rate values measured during the specified time period.

According to the method described above, upon detection of a heart rate abnormality in a state or mode of the wearable electronic device set to measure the heart rate of the user at specified time intervals, the wearable electronic device 201 may continuously measure the heart rate of the user during a specific time. Through this, the wearable electronic device 201 according to an embodiment may effectively provide a notification indicating the heart rate abnormality to the user.

According to an embodiment, the wearable electronic device 201 may include the first sensor 270, the second sensor 280, the processor 220, and the memory 230 storing instructions. According to an embodiment, the instructions may be configured to, when executed by the at least one processor, cause the wearable electronic device to, in a first state of the wearable electronic device set to measure a user's heart rate at specified time intervals, measure, using the first sensor, a first heart rate of the user during a first time. According to an embodiment, the instructions may be configured to, when executed by the at least one processor, cause the wearable electronic device to, in a first state of the wearable electronic device set to, based on identifying that the first heart rate is outside a specified range, measure, using the first sensor, second heart rates of the user during a second time after the first time. According to an embodiment, the instructions may be configured to, when executed by the at least one processor, cause the wearable electronic device to, in a first state of the wearable electronic device set to, based on identifying that a ratio of heart rates outside the specified range among the second heart rates measured during the second time exceeds a specified ratio, output a notification indicating an abnormality in a heart rate of the user.

According to an embodiment, the instructions may be configured to, when executed by the at least one processor, cause the wearable electronic device to, in the first state, determine whether the user is at rest based on the user's movement identified through the second sensor. According to an embodiment, the instructions may be configured to, when executed by the at least one processor, cause the wearable electronic device to measure the first heart rate based on identifying that the user is at rest.

According to an embodiment, the instructions may be configured to, when executed by the at least one processor, cause the wearable electronic device to identify whether a signal to noise SNR of a first biosignal acquired using the first sensor to measure the first heart rate is greater than a specified value. According to an embodiment, the instructions may be configured to, when executed by the at least one processor, cause the wearable electronic device to determine whether the first heart rate is outside the specified range, based on identifying that the SNR of the first biosignal is not greater than the specified value.

According to an embodiment, the instructions may be configured to, when executed by the at least one processor, cause the wearable electronic device to, based on identifying that the first heart rate is outside a specified range, identify a wearing state of the wearable electronic device. According to an embodiment, the instructions may be configured to, when executed by the at least one processor, cause the wearable electronic device to stop measuring the second heart rate, when it is identified that the wearing state does not satisfy a specified condition.

According to an embodiment, the instructions may be configured to, when executed by the at least one processor, cause the wearable electronic device to, based on identifying that the first heart rate exceeds a first reference value related to a high heart rate, identify a movement of the user through the second sensor. According to an embodiment, the instructions may be configured to, when executed by the at least one processor, cause the wearable electronic device to stop measuring the second heart rate, when a sensing value indicating the movement of the user is greater than a specified value.

According to an embodiment, the instructions may be configured to, when executed by the at least one processor, cause the wearable electronic device to, when a ratio of heart rates higher than a first reference value related to a high heart rate among the second heart rates measured during the second time is identified to exceed the specified ratio, provide information indicating a high heart rate of the user through the notification.

According to an embodiment, the instructions may be configured to, when executed by the at least one processor, cause the wearable electronic device to, when a ratio of heart rates lower than a second reference value related to a low heart rate among the second heart rates measured during the second time is identified to exceed the specified ratio, provide information indicating a low heart rate of the user through the notification.

According to an embodiment, the instructions may be configured to, when executed by the at least one processor, cause the wearable electronic device to stop measuring the second heart rate, when a function related to an exercise mode is executed while measuring the second heart rate.

According to an embodiment, the instructions may be configured to, when executed by the at least one processor, cause the wearable electronic device to stop measuring the second heart rate, when a command to measure the user's bio-information different from the heart rate is identified through the first sensor while measuring the second heart rate.

According to an embodiment, the instructions may be configured to, when executed by the at least one processor, cause the wearable electronic device to continuously monitor the user's heart rate using the first sensor after outputting the notification.

According to an embodiment, a method for operating the wearable electronic device 201 may include, in a first state of the wearable electronic device set to measure a user's heart rate at specified time intervals, measuring a first heart rate of the user during a first time using the first sensor 270 included in the wearable electronic device. According to an embodiment, the method for operating the wearable electronic device may include, based on identifying that the first heart rate is outside a specified range, measuring second heart rates of the user during a second time after the first time using the first sensor. According to an embodiment, the method for operating the wearable electronic device may include, based on identifying that a ratio of heart rates outside the specified range among the second heart rates measured during the second time exceeds a specified ratio, outputting a notification indicating an abnormality in a heart rate of the user.

According to an embodiment, measuring the first heart rate may include, in the first state, determining whether the user is at rest based on the user's movement identified through a second sensor 280 included in the wearable electronic device. According to an embodiment, measuring the first heart rate may include measuring the first heart rate based on identifying that the user is at rest.

According to an embodiment, identifying that the first heart rate is outside the specified range may include identifying whether an SNR of a first biosignal acquired using the first sensor to measure the first heart rate is greater than a specified value. According to an embodiment, identifying that the first heart rate is outside the specified range may include determining whether the first heart rate is outside the specified range, based on identifying that the SNR of the first biosignal is not greater than the specified value.

According to an embodiment, the method for operating the wearable electronic device may further include, based on identifying that the first heart rate is outside a specified range, identifying a wearing state of the wearable electronic device. According to an embodiment, the method for operating the wearable electronic device may further include stopping measuring the second heart rate, when it is identified that the wearing state does not satisfy a specified condition.

According to an embodiment, the method for operating the wearable electronic device may further include, based on identifying that the first heart rate exceeds a first reference value related to a high heart rate, identifying a movement of the user through the second sensor. According to an embodiment, the method for operating the wearable electronic device may further include stopping measuring the second heart rate, when a sensing value indicating the movement of the user is greater than a specified value.

According to an embodiment, outputting the notification indicating the abnormality in the heart rate may include, when a ratio of heart rates higher than a first reference value related to a high heart rate among the second heart rates measured during the second time is identified to exceed the specified ratio, providing information indicating a high heart rate of the user through the notification.

According to an embodiment, outputting the notification indicating the abnormality in the heart rate may include, when a ratio of heart rates lower than a second reference value related to a low heart rate among the second heart rates measured during the second time is identified to exceed the specified ratio, providing information indicating a low heart rate of the user through the notification.

According to an embodiment, the method for operating the wearable electronic device may further include stopping measuring the second heart rate, when a function related to an exercise mode is executed while measuring the second heart rate.

According to an embodiment, the method for operating the wearable electronic device may further include continuously monitoring the user's heart rate using the first sensor after outputting the notification.

According to an embodiment, the non-transitory recording medium may store instructions that perform operations of, in a first state of the wearable electronic device 201 set to measure a user's heart rate at specified time intervals, measuring a first heart rate of the user during a first time using the first sensor 270 included in the wearable electronic device, based on identifying that the first heart rate is outside a specified range, measuring second heart rates of the user during a second time after the first time using the first sensor, and based on identifying that a ratio of heart rates outside the specified range among the second heart rates measured during the second time exceeds a specified ratio, outputting a notification indicating an abnormality in a heart rate of the user.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. As used herein, each of such phrases as “A or B”, “at least one of A and B”, “at least one of A or B”, “A, B, or C”, “at least one of A, B, and C”, and “at least one of A, B, or C”, may include any one of, or all possible combinations of the items enumerated together in a corresponding one of the phrases. As used herein, such terms as “1^{st}” and “2^{nd}”, or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, logic, logic block, part, or circuitry. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 1440) including one or more instructions that are stored in a storage medium (e.g., internal memory 1436 or external memory 1438) that is readable by a machine (e.g., the electronic device 1401). For example, a processor (e.g., the processor 1420) of the machine (e.g., the electronic device 1401) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable electronic device comprising:
a first sensor;
a second sensor;
memory, comprising one or more storage media, storing instructions; and
one or more processors communicatively coupled to the first sensor, the second sensor, and the memory,
wherein the instructions, when executed by the one or more processors individually or collectively, cause the wearable electronic device to:
in a first state of the wearable electronic device set to measure a heart rate of a user at specified time intervals, measure, using the first sensor, a first heart rate of the user during a first time,
based on identifying that the first heart rate is outside a specified range, measure, using the first sensor, second heart rates of the user during a second time after the first time, and
based on identifying that a ratio of heart rates outside the specified range among the second heart rates measured during the second time exceeds a specified ratio, output a notification indicating an abnormality in the heart rate of the user.

2. The wearable electronic device of claim 1, wherein the instructions, when executed by the one or more processors individually or collectively, further cause the wearable electronic device to:
in the first state, determine whether the user is at rest based on a user's movement identified through the second sensor, and
measure the first heart rate based on identifying that the user is at rest.

3. The wearable electronic device of claim 1, wherein the instructions, when executed by the one or more processors individually or collectively, further cause the wearable electronic device to:
identify whether a signal to noise ratio (SNR) of a first biosignal acquired using the first sensor to measure the first heart rate is greater than a specified value, and
determine whether the first heart rate is outside the specified range, based on identifying that the SNR of the first biosignal is less than the specified value.

4. The wearable electronic device of claim 1, wherein the instructions, when executed by the one or more processors individually or collectively, further cause the wearable electronic device to:
based on identifying that the first heart rate is outside a specified range, identify a wearing state of the wearable electronic device, and
stop the measuring of the second heart rates, when it is identified that the wearing state does not satisfy a specified condition.

5. The wearable electronic device of claim 1, wherein the instructions, when executed by the one or more processors individually or collectively, further cause the wearable electronic device to:
based on identifying that the first heart rate exceeds a first reference value related to a high heart rate, identify a movement of the user through the second sensor, and
stop the measuring of the second heart rates, when a sensing value indicating the movement of the user is greater than a specified value.

6. The wearable electronic device of claim 1, wherein the instructions, when executed by the one or more processors individually or collectively, further cause the wearable electronic device to:
when a ratio of heart rates higher than a first reference value related to a high heart rate among the second heart rates measured during the second time is identified to exceed the specified ratio, provide information indicating a high heart rate of the user through the notification.

7. The wearable electronic device of claim 1, wherein the instructions, when executed by the one or more processors individually or collectively, further cause the wearable electronic device to:
when a ratio of heart rates lower than a second reference value related to a low heart rate among the second heart rates measured during the second time is identified to exceed the specified ratio, provide information indicating a low heart rate of the user through the notification.

8. The wearable electronic device of claim 1, wherein the instructions, when executed by the one or more processors individually or collectively, further cause the wearable electronic device to:
stop the measuring of the second heart rates, when a function related to an exercise mode is executed while measuring the second heart rates.

9. The wearable electronic device of claim 1, wherein the instructions, when executed by the one or more processors individually or collectively, further cause the wearable electronic device to:
stop the measuring of the second heart rates, when a command to measure bio-information of the user different from the heart rate is identified through the first sensor while measuring the second heart rates.

10. The wearable electronic device of claim 1, wherein the instructions, when executed by the one or more processors individually or collectively, further cause the wearable electronic device to:
continuously monitor the heart rate of the user using the first sensor after outputting the notification.

11. A method performed by a wearable electronic device, the method comprising:
in a first state of the wearable electronic device set to measure a heart rate of a user at specified time intervals, measuring, by the wearable electronic device using a first sensor included in the wearable electronic device, a first heart rate of the user during a first time;
based on identifying that the first heart rate is outside a specified range, measuring, by the wearable electronic device using the first sensor, second heart rates of the user during a second time after the first time; and
based on identifying that a ratio of heart rates outside the specified range among the second heart rates measured during the second time exceeds a specified ratio, outputting, by the wearable electronic device, a notification indicating an abnormality in the heart rate of the user.

12. The method of claim 11, wherein the measuring of the first heart rate comprises:
in the first state, determining, by the wearable electronic device, whether the user is at rest based on movement of the user identified through a second sensor included in the wearable electronic device; and
measuring, by the wearable electronic device, the first heart rate based on identifying that the user is at rest.

13. The method of claim 11, wherein the identifying that the first heart rate is outside the specified range comprises:
identifying, by the wearable electronic device, whether a signal to noise ratio (SNR) of a first biosignal acquired using the first sensor to measure the first heart rate is greater than a specified value; and
determining, by the wearable electronic device, whether the first heart rate is outside the specified range, based on identifying that the SNR of the first biosignal is less than the specified value.

14. The method of claim 11, further comprising:
based on identifying that the first heart rate is outside a specified range, identifying, by the wearable electronic device, a wearing state of the wearable electronic device; and
stopping, by the wearable electronic device, the measuring of the second heart rates, based on identifying that the wearing state does not satisfy a specified condition.

15. The method of claim 11, further comprising:
based on identifying that the first heart rate exceeds a first reference value related to a high heart rate, identifying, by the wearable electronic device, a movement of the user through a second sensor included in the wearable electronic device; and
stopping, by the wearable electronic device, the measuring of the second heart rates, based on a sensing value indicating the movement of the user is greater than a specified value.

16. The method of claim 11, further comprising:
based on a ratio of heart rates higher than a first reference value related to a high heart rate among the second heart rates measured during the second time is identified to exceed the specified ratio, providing, by the wearable electronic device, information indicating a high heart rate of the user through the notification.

17. The method of claim 11, further comprising:
based on a ratio of heart rates lower than a second reference value related to a low heart rate among the second heart rates measured during the second time being identified to exceed the specified ratio, providing, by the wearable electronic device, information indicating a low heart rate of the user through the notification.

18. The method of claim 11, further comprising:
stopping, by the wearable electronic device, the measuring of the second heart rates, based on a function related to an exercise mode being executed while measuring the second heart rates.

19. One or more non-transitory computer-readable storage media storing one or more computer programs including computer-executable instructions that, when executed by one or more processors of a wearable electronic device individually or collectively, cause the wearable electronic device to perform operations, the operations comprising:
in a first state of the wearable electronic device set to measure a heart rate of a user at specified time intervals, measuring, by the wearable electronic device using a first sensor included in the wearable electronic device, a first heart rate of the user during a first time;
based on identifying that the first heart rate is outside a specified range, measuring, by the wearable electronic device using the first sensor, second heart rates of the user during a second time after the first time; and
based on identifying that a ratio of heart rates outside the specified range among the second heart rates measured during the second time exceeds a specified ratio, outputting, by the wearable electronic device, a notification indicating an abnormality in the heart rate of the user.

20. The one or more non-transitory computer-readable storage media of claim 19, wherein the measuring of the first heart rate comprises:
in the first state, determining, by the wearable electronic device, whether the user is at rest based on movement of the user identified through a second sensor included in the wearable electronic device; and
measuring, by the wearable electronic device, the first heart rate based on identifying that the user is at rest.
